(19) United States
(12) Patent Application Publication
(10) Pub. No.: US 2016/0353733 A1
(43) Pub. Date: Dec. 8, 2016

(54) METHODS FOR REDUCING AIRBORNE BACTERIA AND MYCETES AND APPARATUS FOR THE SAME

(71) Applicant: Philadelphia University, Philadelphia, PA (US)

(72) Inventors: Diana R. Cundell, Philadelphia, PA (US); Brian R. George, Pottstown, PA (US)

(21) Appl. No.: 14/990,067

(22) Filed: Jan. 7, 2016

Related U.S. Application Data
(63) Continuation of application No. 13/768,806, filed on Feb. 15, 2013, now abandoned.
Application No. 13/768,806 is a continuation-in-part of application No. 12/705,843, filed on Feb. 15, 2010, now abandoned.
Application No. 12/705,843 is a continuation-in-part of application No. 13/052,592, filed on Mar. 21, 2011, now abandoned.
Application No. 13/052,592 is a continuation-in-part of application No. 13/112,252, filed on May 20, 2011, now abandoned.
(60) Provisional application No. 61/600,882, filed on Feb. 20, 2012.

(51) Int. Cl. A01N 31/16; A61L 9/16; F24F 3/16; A01N 25/34

(52) U.S. Cl. CPC A01N 31/16; A01N 25/34; A61L 9/16; F24F 3/16; A61L 2209/14; F24F 2003/1653; F24F 2003/1664

(57) ABSTRACT

A method for removing airborne bacteria and airborne mycetes from indoor air comprises imparting microbiocidal and microbiostatic properties to fabric by treating the fabric with one or more naturally occurring biocides, positioning the treated fabric in a sheet-like orientation, and blowing indoor air through the fabric.

### CROSS-REFERENCE TO RELATED PATENT APPLICATIONS

This patent application is a continuation of U.S. patent application Ser. No. 13/678,806 entitled Methods for Reducing Airborne Bacteria and Mycetes and Apparatus for the Same” filed 14 Feb. 2013, which application claims the priority of benefit under 35 USC 119 and 35 USC 120 of provisional U.S. patent application Ser. No. 61/600,882 entitled “Methods and Apparatus for Reduction of Airborne Bacteria and Mycetes” filed 20 Feb. 2012, and U.S. patent application Ser. No. 12/705,843 filed 15 Feb. 2010; and U.S. patent application Ser. No. 13/052,592 filed 21 Mar. 2011; and U.S. patent application Ser. No. 13/112,252 filed 20 May 2011. The disclosures of these three pending United States utility patent publications are hereby incorporated by reference in their entirety.

### BACKGROUND OF THE INVENTION

Field of the Invention

This invention relates to reduction of airborne bacteria and mycetes.

### SUMMARY OF THE INVENTION

In one of its aspects, this invention provides a method for removing airborne bacteria and airborne mycetes from indoor air, where the method includes imparting microbicidal and microbiostatic properties to a fabric by treating the fabric with one or more naturally occurring biocides; positioning the fabric, as treated, preferably in a sheet-like orientation and blowing indoor air through the fabric. In the practice of the method, preferably at least one of the naturally occurring biocides is eugenol. The fabric is preferably a knit and most preferably a circular knit, with the fabric comprising cotton and most preferably being all cotton.

In another aspect of the invention, the imparting of microbiocidal and microbiostatic properties to the fabric further preferably involves immersing the fabric in a solution of glyoxal, eugenl, polyvinyl alcohol and water, squeezing the solution from the fabric, curing the wetted fabric under heat, and drying the cured fabric.

In the practice of another aspect of the invention, the mass of the solution is preferably about ten times the mass of the fabric. The solution preferably comprises preferably about 10 grams of glyoxal per liter of solution, preferably about 1 gram of eugenol per liter of solution, and preferably about 1 gram of polyvinyl alcohol per liter of solution.

The squeezing of the fabric is preferably performed by wringing the fabric between rolls of wringer.

The bacteria removed from the air through practice of this aspect of the invention include Staphylococci and Bacillus. The mycetes removed from the air in the practice of this aspect of the invention include Alternaria, Aspergillus, Cladosporium, Penicillium and various yeast species.

### BRIEF DESCRIPTION OF THE DRAWINGS

FIG. 1 is a bar graph showing colony forming units of bacteria (“CFU”), on the ordinate of the bar graph of bacteria, recovered in accordance with the invention over a six week period discussed in the specification hereinbelow. The abscissa of FIG. 1 presents the time scale in weeks.

FIG. 2 is a bar graph similar to FIG. 1, showing on the ordinate of FIG. 2 colony forming units (“CFU”) of mycetes, recovered in accordance with the invention over the six week period described in the specification below. The abscissa of FIG. 2 presents the same time scale in weeks as the abscissa of FIG. 1.

FIG. 3 is a bar graph showing colony forming units of bacteria and mycetes (“CFU”), on the ordinate of the bar graph, recovered in accordance with the invention over the six week period discussed in this specification hereinbelow. The abscissa of FIG. 3 presents the same time scale in weeks as the abscissa of FIGS. 1 and 2.

FIG. 4 is a bar graph showing colony forming units of bacilli (“CFU”), on the ordinate of the bar graph, recovered over the six week period discussed in the specification hereinbelow. The abscissa of FIG. 4 presents the time scale in weeks the same as the abscissa of FIGS. 1, 2 and 3.

FIG. 5 is a bar graph showing colony forming units of staphylococci (“CFU”) on the ordinate of the bar graph of bacteria recovered over a six week period discussed in this specification hereinbelow. The abscissa of FIG. 5 presents the same time scale in weeks as the abscissa of FIGS. 1 through 4.

### DETAILED DESCRIPTION OF THE INVENTION

The invention will now be described in greater detail with reference to the preferred embodiments which follow. It should be appreciated, however, these embodiments are for illustrative purposes only and are not meant to limit the invention, which are defined by the claims.

This invention provides apparatus, methods, fabric, and fabric treatment processes for removing airborne bacteria and airborne mycetes from indoor air by imparting microbicidal and microbiostatic properties to fabric, specifically by treating the fabric with one or more naturally occurring biocides, positioning the fabric preferably in a sheet-like orientation, and preferably blowing indoor air through the fabric. Preferably, at least one of the naturally occurring biocides is eugenol. The fabric is preferably a knit, most preferably a circular knit, and preferably includes cotton, and most preferably is all cotton.

Microbicidal and microbiostatic properties are imparted to the fabric by preferably immersing the fabric in a solution of glyoxal, eugenol, polyvinyl alcohol and water, squeezing the solution out of the fabric, curing the wetted fabric under heat, and drying the cured fabric.

Desirably, the mass of the solution is about ten times the mass of the fabric. The solution preferably comprises about 10 grams of glyxol per liter of solution, about 1 gram of eugenol per liter of solution, and about 1 gram of polyvinyl alcohol per liter of solution. The squeezing operation on the fabric is preferably performed by wringing the fabric between rolls of a wringer.

The airborne bacteria removed from indoor air using the invention include Staphylococci and Bacillus. The airborne mycetes removed from indoor air using the invention include Alternaria, Aspergillus, Cladosporium, Pennicillium and yeast species.

Efficacy of the invention has been demonstrated through a field test in the United States Veterans Administration Hospital of Southeastern Pennsylvania, in which four rooms having the following characteristics were selected in the Veterans Hospital: All of the rooms were in a similar location, along a corridor on the first floor of the Veterans Administration Hospital. Each room had a closed air system with only one air intake and one outlet vent. Each of the four rooms were of similar dimensions. All four of the rooms were used as consulting rooms, where physicians saw patients having a variety of infectious and non-infectious conditions.

In each room two locations were selected for sampling. One sampling was close to a modular unit for improving indoor air quality of the type generally disclosed in the '843 patent application as referenced above. The second location chosen for sampling was near the entry point or central area of the room.

For each room the sample locations were as follows:
1. Room 1
   a. Telephone (next to a modular unit similar to that disclosed in '843)
   b. Work desk (near entry point of room)
2. Room 2
   a. Wall mounted X-ray (next to a modular unit similar to that disclosed in '843)
   b. Work desk (near entry point of room)
3. Room 3
   a. Tray table (next to a modular unit similar to that disclosed in '843)
   b. Work desk (near entry point of room)
4. Room 4
   a. Under modular unit as disclosed in '843
   b. Work desk (central area of room)

The modular units were installed and two air quality samples taken in each room on the day of installation (day 0) and then one, two, three and four weeks after installation. After week four, the units were switched off.

The air quality samples were obtained using sedimentation plates using the technique disclosed in the '592 patent application referenced above. The three agars selected, namely nutrient agar (all purpose medium), Sabouraud-dextrose agar (fungal isolation), and sheep blood agar (pathogens particularly staphylococci and bacilli), were left open at two locations in each room for a period of one hour then closed, and the plates incubated at 30° C. for between 24 and 48 hours. After this time both the number and type of colony on each plate were counted. Those which were not able to be determined by this method were gram stained and examined microscopically.

Data obtained upon installation and after one week of usage of the modular units was pooled and used as baseline air quality for each room. This provided four values in total for each location. Values are given as CFU (colony forming units) of bacteria/fungi recovered after the one hour sedimentation collection had ceased.

Week 6 data was at issue for Rooms 1 and 2, as the modular units were accidentally switched on in these rooms by staff after week 5 sampling. As a result room 1 and 2 data have been removed from the calculations in all week 6 data.

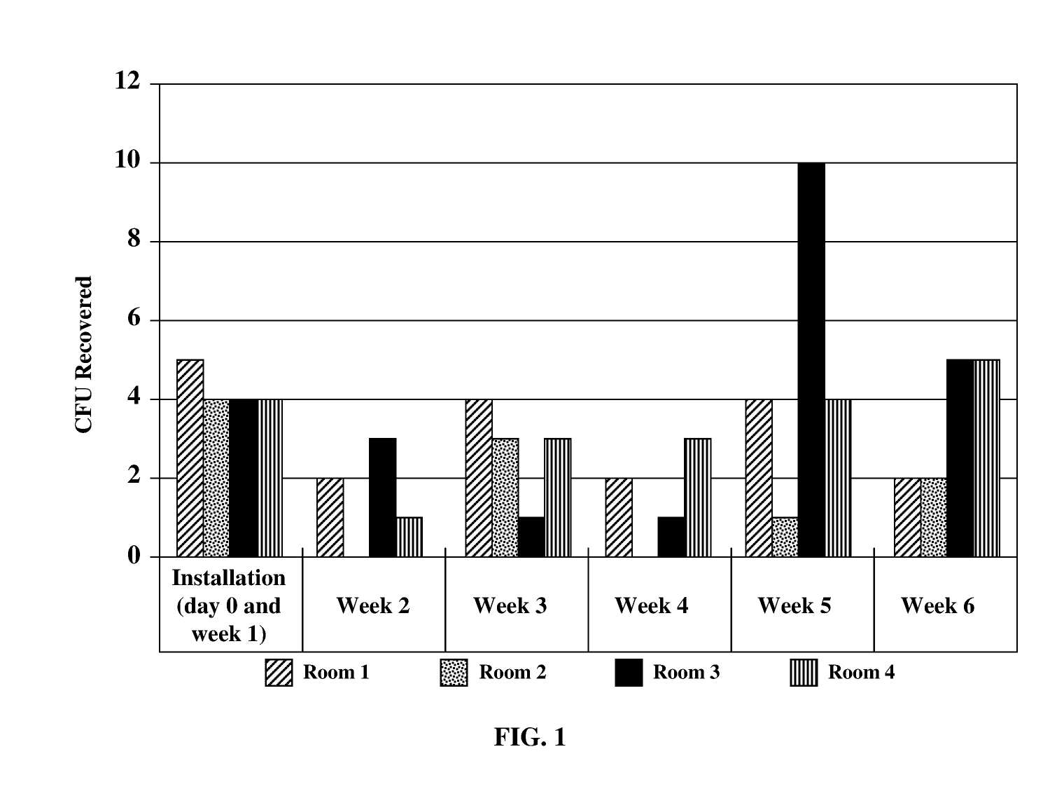

Bacterial numbers were low across the four rooms but reduced still further from a mean value of 4.3 recovered per plate, prior to the modular unit in the rooms, to mean values of 1.5, 2.8 and 1.5 during weeks 2, 3 and 4 of the testing cycle, respectively. This corresponded to decreases of 35% to 65%, with a mean decrease across weeks 2 through 4 of 55%. After the modular unit was switched off (week 4), numbers increased to an average of 4.8 (week 5) and 5.0 (week 6), commensurate with pre-testing values. FIG. 1 shows the results in colony forming units of bacteria recovered over the six week experimental period.

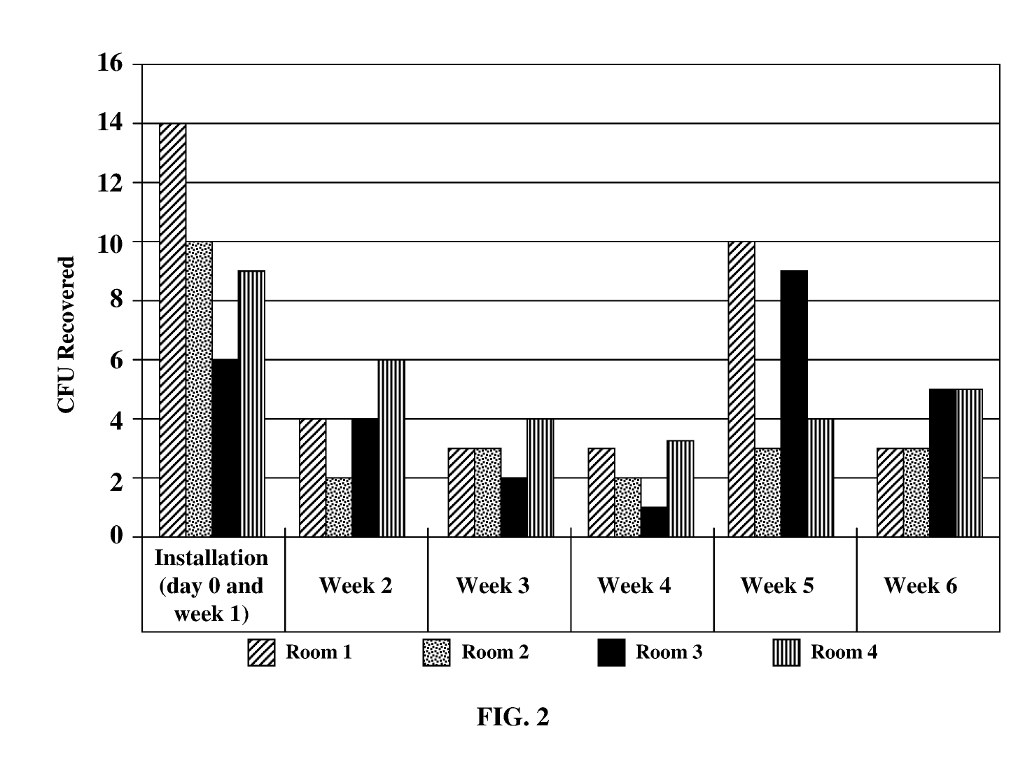

Mycetes were the dominant airborne microbe across the four rooms with a mean value of 9.8 recovered per plate prior to installation of the modular unit in the rooms. Airborne numbers decreased with time to mean values of 4.0, 3.0 and 2.3 during weeks 2, 3 and 4 respectively. This corresponded to 59% to 76% of the original mean count or a 68.4% decrease in numbers. After the modular units were switched off (week 4) microbes recovered increased to an average of 6.5 (week 5) and 5.0 (week 6). These did not return to pre-testing values but did show recovery from the trial period. FIG. 2 shows the colony forming units of mycetes recovered over the six week period.

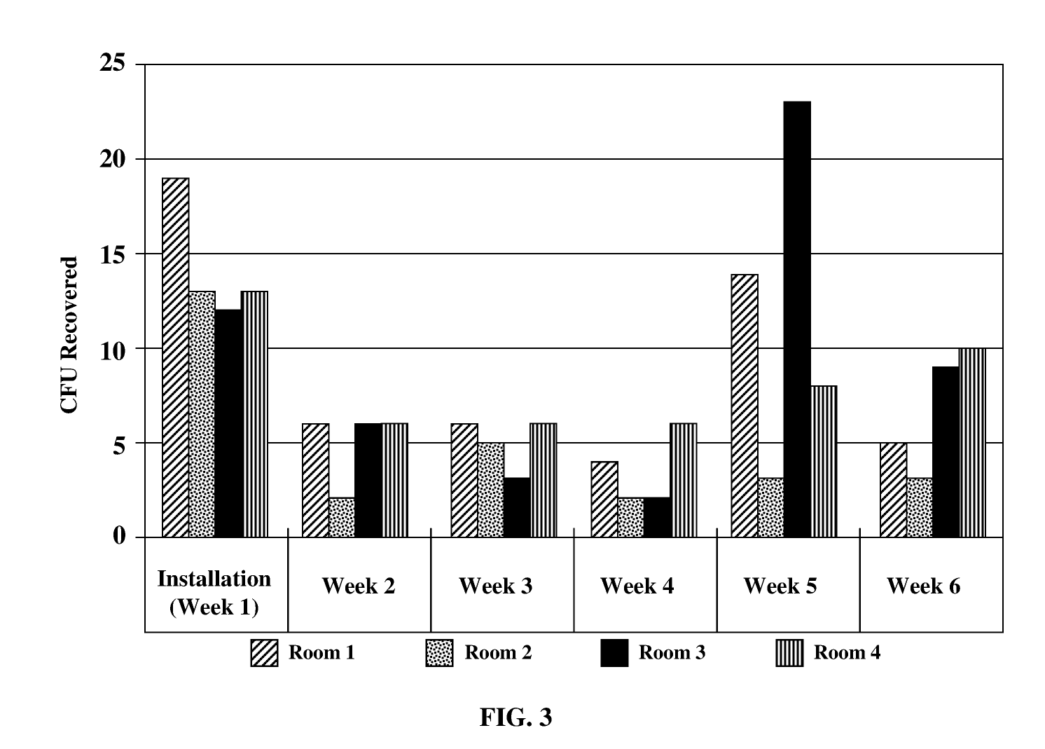

Combined values for bacteria and mycetes were 14.3 recovered per plate prior to the modular unit installation. These decreased to mean values of 3.5, 5.0 and 3.0 during weeks 2, 3 and 4 respectively. This corresponded to a 65.0% to 75.5% decrease in numbers across weeks 2-4. After the modular units were switched off (week 4) numbers increased to an average of 12.0 (week 5) and 10.0 (week 6), which was commensurate with pre-testing values. These data are shown in FIG. 3.

Two types of bacteria, namely Staphylococci and Bacillus species, and five different types of mycetes, namely Alternaria, Aspergillus, Cladosporium, Penicillium and yeast species, were recovered as the dominant microbes in the hospital consulting rooms.

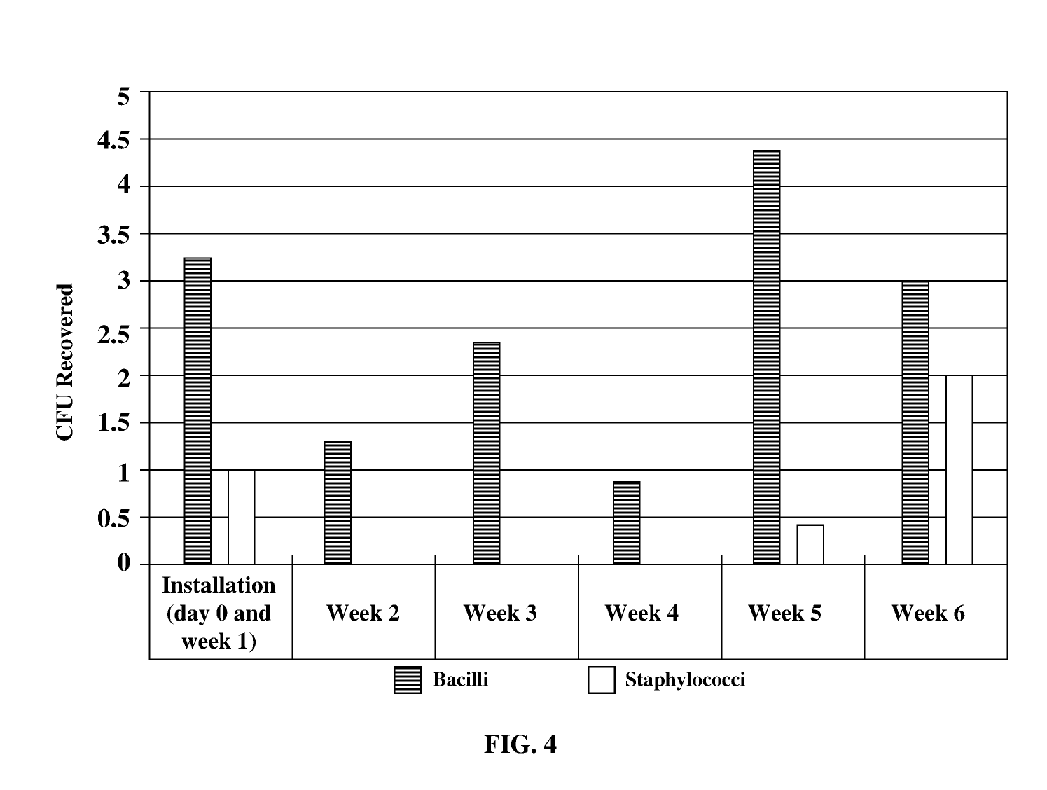

Bacilli were the dominant microbe across the four rooms and the 3.2 recovered per plate prior to the modular unit installation decreased to mean values of 1.3, 2.4 and 0.9 during weeks 2, 3 and 4 of the testing cycle, respectively. This corresponded to decrease in numbers across weeks 2 through 4 of 25% to 72%, with a mean of 53.3% across the three testing weeks. After the modular units were switched off (week 4), numbers increased to an average of 4.3 (week 5) and 3.0 (week 6), commensurate with initial values. These data are shown in FIG. 4.

Few Staphylococci were recovered from the consultation rooms when the modular units were installed but any circulating colonies were removed completely (100%) during the trial period and reappeared when the modular units were switched off and removed (weeks 5 and 6)

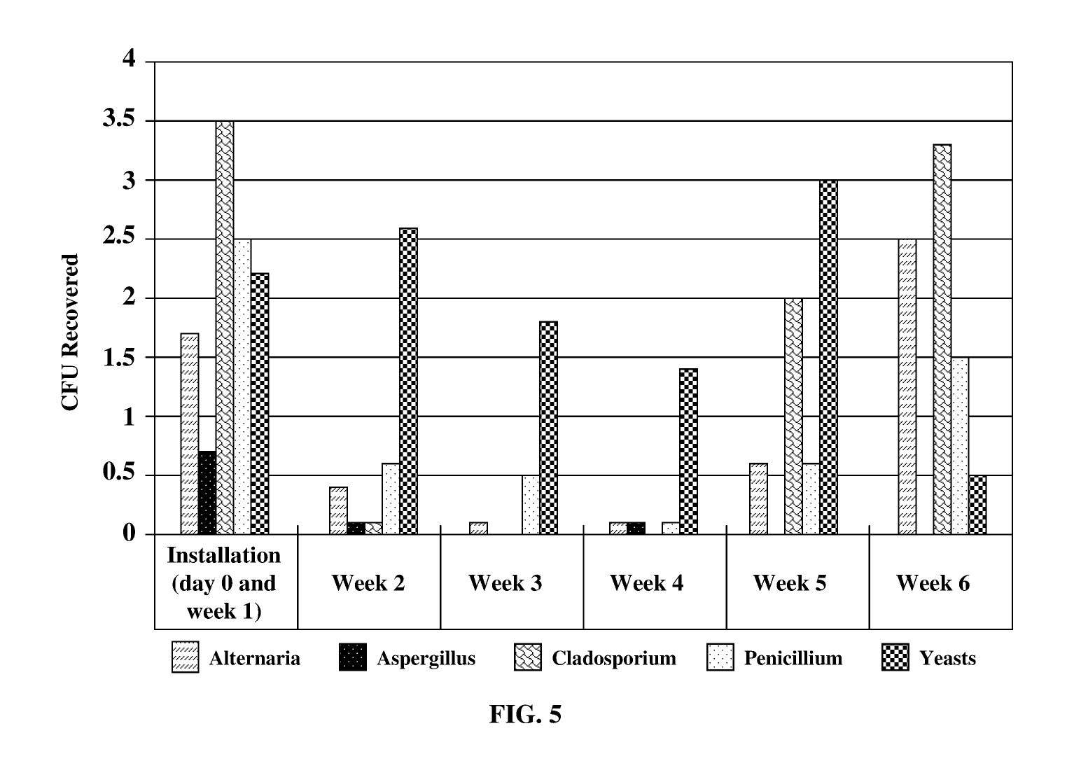

For the mycetes, several species were dominant in the hospital consulting rooms, namely Cladosporium, Penicillium and yeast species, as can be seen in FIG. 5. Cladosporia were recovered at a mean rate of 3.5 per plate prior the installation of the modular units and decreased to mean values of 0.1, 0.0 and 0.0 during weeks 2, 3 and 4 respectively. This corresponded to decrease in numbers across weeks 2-4 of 97-100% with a mean of 99% across the three testing weeks. After the modular units were switched off (week 4), numbers increased to an average of 2.0 (week 5) and 3.3 (week 6), commensurate with prior levels.

Penicillium species were the second most dominant mold isolated with mean values of 2.5 colonies per plate prior to installation of the modular units. Recovered colonies fell to 0.6, 0.6 and 0.1 during weeks 2, 3 and 4 of the cycle, corresponding to a percentage decrease of 76-96%, or mean percentage decrease of 82.7% across the three weeks. After the modular units were switched off (week 4), numbers increased to an average of 0.6 (week 5) and 1.5 (week 6), which showed a trend towards the initial values. Yeast species were also frequently recovered (mean of 2.2 per plate during the installation phase of the modular units) but fell more slowly during the testing phase, taking two weeks (week 3) to fall significantly (36.4%). They also rapidly returned to pre-test levels of recovery after the modular unit was switched off.

Two other mycetes were also recovered from the hospital consulting rooms, namely Alternaria and Aspergillus. Alternaria were recovered at a mean rate of 1.7 per plate prior during the installation of the modular units and decreased to mean values of 0.4, 0.1 and 0.1 during weeks 2, 3 and 4 of the cycle, respectively. This corresponded to a decrease in numbers across weeks 2 through 4 of 76% to 94% with a mean of 88% across the three testing weeks. After the modular units were switched off (week 4), numbers increased to an average of 0.6 (week 5) and 2.5 (week 6), commensurate with pre-testing values.

Aspergillus were infrequently recovered (mean rate of 0.7 per plate prior during the installation phase), decreased to mean values of 0.1, 0 and 0 during weeks 2, 3 and 4 of the cycle respectively and did not reappear after the modular units were switched off. These data are shown in FIG. 5.

The modular units are effective for abrogating the levels of airborne bacteria and molds, as evidenced by the following findings:

1) There was a decrease of 65-75% in total airborne microbes during the the trial (weeks 2-4).

2) The modular units were effective against bacteria (35-65%; mean 55%) with both Staphylococci and Bacilli equally affected.

3) The modular units were effective against molds (59-76%; mean 68.4%). Efficacy against the airborne molds from highest to lowest was Cladosporium (97-100% reduction; mean 99%)>Alternaria (76-94% reduction; mean 88%)>Penicillium (76-96%; mean percentage 82.7%)>Yeast (25-41%; mean 36.4%). Aspergillus species were infrequently isolated.

4) Levels of both bacterial species (Staphylococci and Bacilli) and four of the five molds (Alternaria, Aspergillus, Cladosporium and yeast species) returned to prior values when the modular units were switched off after week 4. Penicillium was close to pre-test values.

These findings are important as they demonstrate the invention is:

1. Has rapid and sustained efficacy.

2. Has a broad spectrum of activity.

3. Is of benefit in locations with sick building syndrome, in that Aspergillus, Alternaria and Penicillium are proven causes.

4. Sporicidal, namely active against Bacillus species.

While invention has been described in detail with respect to the preferred embodiments and other embodiments for practice of the invention, it is to be understood that the scope of the patent to be issued from this application is to be construed according to the scope of the claims and equivalents thereto, with the claims being as set forth below.

I claim the following:

1) A method for removing airborne bacteria and airborne mycetes from indoor air comprising the steps of:
a) imparting microbicidal and microbiostatic properties to fabric by treating the fabric with one or more naturally occurring biocides;
b) positioning the treated fabric in an orientation to have air blown therethrough; and
c) blowing indoor air through the fabric.

2) The method of claim 1 wherein at least one of the naturally occurring biocides is eugenol.

3) The method of claim 1 wherein the fabric is a knit.

4) The method of claim 3 wherein the knit is a circular knit.

5) The method of claim 1 wherein the fabric comprises cotton.

6) The method of claim 4 wherein the fabric is all cotton.

7) The method of claim 3 wherein the fabric is cotton.

8) The method of claim 1 wherein the step of imparting microbicidal and microbiostatic properties to the fabric further comprises:
a) immersing the fabric in a solution of glyoxal, eugenol, polyvinyl alcohol and water;
b) squeezing the solution out of the fabric
c) curing the wetted fabric under heat; and
d) drying the cured fabric.

9) The method of claim 8 wherein the mass of the solution is about ten times the mass of the fabric.

10) The method of claim 9 wherein the solution comprises about 10 grams of glyxol per liter of solution, about 1 gram of eugenol per liter of solution and about 1 gram of polyvinyl alcohol per liter of solution.

11) The method of claim 8 wherein squeezing is performed by wringing the fabric between rolls of a wringer.

12) The method of claim 1 wherein the airborne bacteria include Staphylococci and Bacillus.

13) The method of claim 1 wherein the airborne mycetes include Alternaria, Aspergillus, Cladosporium, Penicillium and yeast species.

14) The method of claim 7 wherein the step of imparting microbicidal and microbiostatic properties to the fabric further comprises:
a) immersing the fabric in an aqueous solution of glyoxal, eugenol, and polyvinyl alcohol;
b) squeezing the solution out of the fabric
c) curing the wetted fabric under heat; and
d) drying the cured fabric.

15) The method of claim 14 wherein the mass of the solution is ten times the mass of the fabric.

16) The method of claim 15 wherein the solution comprises about 10 grams of glyxol per liter of solution, about 1 gram of eugenol per liter of solution and about 1 gram of polyvinyl alcohol per liter of solution.

17) The method of claim 16 wherein squeezing is performed by wringing the fabric between rolls of a wringer.

18) The method of claim 16 wherein the airborne bacteria include Staphylococci and Bacillus and the airborne mycetes include Alternaria, Aspergillus, Cladosporium, Penicillium and yeast species.

19) The method of claim 9 whereon the solution comprises about 10 grams of glyxol, 1 gram of eugenol, and 1 gram of polyvinyl alcohol per liter of water.
